(19) United States
(12) Patent Application Publication
(10) Pub. No.: US 2015/0209372 A1
(43) Pub. Date: Jul. 30, 2015

(54) COMBINATION TREATMENT FOR ALCOHOL DEPENDENT PATIENTS

(71) Applicant: Jeffrey Shelton, Farmington, CT (US)

(72) Inventor: Jeffrey Shelton, Farmington, CT (US)

(21) Appl. No.: 14/165,958

(22) Filed: Jan. 28, 2014

(51) Int. Cl. A61K 31/5517; A61K 31/5513; A61K 31/145

(52) U.S. Cl. CPC A61K 31/5517; A61K 31/145; A61K 31/5513

(57) ABSTRACT

Described herein is the use of a combination of disulfiram and a benzodiazepine for the treatment of individuals suffering from severe alcohol use disorder with high utilization of inpatient detoxification. The combination agent reduces a recurrence of hospitalization and reduces cravings in this extremely difficult to treat population.

### FIELD OF THE DISCLOSURE

The present disclosure is related to combination treatments for alcohol dependent patients, particularly patients with a history of inpatient detoxification.

### BACKGROUND

Alcohol Dependence (AD) is major public health problem which affects patients, families, and communities as well as creates significant financial healthcare expenditure and costly utilization of inpatient resources. Of particular importance in the treatment of AD patients is the reduction of cravings so that patients can maintain sobriety on an outpatient basis.

Currently, disulfiram is used in the treatment of alcoholism. Disulfiram blocks the processing of alcohol by the body by inhibiting acetaldehyde dehydrogenase and causing an unpleasant reaction when alcohol is consumed. Because disulfiram does not reduce alcohol cravings, a problem with the use of disulfiram is patient compliance.

What is needed are treatments for AD that reduce patient cravings and also allow patients to remain sober on an outpatient basis.

### BRIEF SUMMARY

In an aspect, a method of decreasing a recurrence of hospitalization of an individual suffering from severe alcohol use disorder with high utilization of inpatient detoxification comprises administering daily to the individual a combination of disulfiram and a benzodiazepine in amounts sufficient to reduce or eliminate cravings for alcohol and maintain sobriety, wherein severe alcohol use disorder with high utilization of inpatient detoxification is defined as two or more inpatient detoxifications within a 6 month period immediately preceding treatment, and wherein a decrease in the recurrence of hospitalization is one or less hospitalizations in the 3 month period following the start of treatment.

In another aspect, a method of decreasing cravings for alcohol in an individual suffering from severe alcohol use disorder with high utilization of inpatient detoxification comprises administering daily to the individual a combination of disulfiram and a benzodiazepine in amounts sufficient to reduce or eliminate cravings for alcohol and to maintain sobriety, wherein severe alcohol use disorder with high utilization of inpatient detoxification is defined as two or more inpatient detoxifications within a 6 month period immediately preceding treatment, and wherein cravings for alcohol are measured on a Penn Alcohol Cravings Scale of 0 to 30.

The above-described and other features will be appreciated and understood by those skilled in the art from the following detailed description and appended claims.

### DETAILED DESCRIPTION

Described herein is a combination of disulfiram and a benzodiazepine which can be used to treat patients with AD, particularly patients with severe alcohol use disorder with high utilization of inpatient detoxification. Patients with a high frequency of inpatient detoxification, such as 2 to 7 hospitalizations in a 6 month period, are often referred to as community hospital “super-users”, and these patients create a costly utilization of inpatient resources. The goal of treatment is to allow these super-users to maintain sobriety on an outpatient basis, which has proven to be extremely difficult to achieve. It has been unexpectedly shown herein that the combination of disulfiram and a benzodiazepine such as clonazepam can successfully reduce cravings in this particularly challenging patient population and allows super-users to function on an outpatient basis.

The combination of disulfiram and a benzodiazepine was disclosed, for example, in U.S. Pat. No. 5,140,032. In Example 1, study patients were defined as “patients whose long-time alcohol abuse persisted despite multiple hospitalizations and participation in 28-day treatment programs.” U.S. Pat. No. 5,140,032 does not provide enough specificity to identify these patients as super-users as defined herein. In addition, while improvements in sobriety and therapy compliance were reported, there was no indication that future hospitalizations were reduced in the patient population that was studied. WO 2004/054570 also describes the combination of disulfiram and a benzodiazepine to treat alcohol dependence, but also fails to identify the usefulness of the combination therapy for alcohol super-users. It is noteworthy that despite the disclosures of U.S. Pat. No. 5,140,032 and WO 2004/054570, it does not appear that the combination of disulfiram and a benzodiazepine has been successfully marketed for the treatment of alcoholism.

Given the particular challenges of treating alcohol super-users and the lack of a successful treatment for such patients, one would not have expected the combination of disulfiram and a benzodiazepine to be as successful as shown herein. There are several explanations why alcohol super-users are very difficult to treat. One theory, labeled the “kindling” effect, suggests that repeated bouts of withdrawal creates neurobiological sensitivity and susceptibility to development of alcohol withdrawal symptoms, thereby even a brief or low dose relapse can lead to significant withdrawal symptoms and need for medical detoxification. In addition, the negative psychological effects of withdrawal extend beyond the actual physiological withdrawal syndrome treated during a hospitalization. Patients with multiple withdrawal episodes (aka “super users”) experience heightened states of withdrawal related anxiety and malaise after hospitalization which increases the risk for relapse because alcohol relieves their symptoms. Multiple withdrawal episodes expose patients to repeated bouts of positive reinforcement that alcohol will relieve withdrawal related dysphoria which further increases risk for relapse.

In an embodiment, a method of decreasing a recurrence of hospitalization of an individual suffering from severe alcohol use disorder with high utilization of inpatient detoxification comprises administering daily to the individual a combination of disulfiram and a benzodiazepine in amounts sufficient to reduce or eliminate cravings for alcohol and maintain sobriety, wherein severe alcohol use disorder with high utilization of inpatient detoxification is defined as two or more inpatient detoxifications within a 6 month period immediately preceding treatment, and wherein a significant decrease in cravings, relapse, and need for hospitalization is seen following the start of treatment. In an aspect, a decrease in the recurrence of hospitalization is one or less hospitalizations in the 3 month period following the start of treatment. In one embodiment, administering is on an outpatient basis wherein the patient lives outside of the treatment facility, but may visit the facility once or twice per day for administration of medications, for example. In an aspect, there is a decrease in cravings for alcohol with patients going from 20-30/30 on the Penn Alcohol Cravings Scale prior to treatment to 0-5/30 on Penn Alcohol Cravings Scale one week after starting treatment.

In another embodiment, a method of decreasing cravings for alcohol in an individual suffering from severe alcohol use disorder with high utilization of inpatient detoxification comprises administering daily to the individual a combination of disulfiram and a benzodiazepine in amounts sufficient to reduce or eliminate cravings for alcohol and to maintain sobriety, wherein severe alcohol use disorder with high utilization of inpatient detoxification is defined as two or more inpatient detoxifications within a 6 month period immediately preceding treatment, and wherein cravings for alcohol are measured on a Penn Alcohol Cravings Scale of 0 to 30.

As used herein, the term “alcohol craving” includes but is not limited to a physiological-based and/or psychological-based desire for alcohol, for example, alcoholic beverages.

According to the DSM-5, severe alcohol use disorder is defined as the following:

A problematic pattern of alcohol use leading to clinically significant impairment of distress as manifested by at least six (versus just two for mild disorder or 4 for moderate disorder) of the following, occurring within a 12-month period:
1) Alcohol is often taken in larger amounts or over a longer period than was intended;
2) There is a persistent desire or unsuccessful efforts to cut down or control alcohol use;
3) A great deal of time is spent in activities necessary to obtain alcohol, use alcohol, or recover from its effects;
4) Craving, or strong desire or urge to use alcohol;
5) Recurrent alcohol use resulting in a failure to fulfill major role obligations at work, school, or home;
6) Continued alcohol use despite having persistent or recurrent social or interpersonal problems caused or exacerbated by the effects of alcohol;
7) Important social, occupational, or recreational activities are given up or reduced because of alcohol use;
8) Recurrent alcohol use in situations in which it is physically hazardous;
9) Alcohol use is continued despite knowledge of having persistent or recurrent physical or psychological problem that is likely to have been caused or exacerbated by alcohol;
10) Tolerance, as defined by either of the following: a) A need for markedly increased amounts of alcohol to achieve intoxication or desired effect or b) A markedly diminished effect with continued use of the same amount of alcohol; and
11) Withdrawal, as manifested by either of the following: a) The characteristic withdrawal syndrome for alcohol or b) Alcohol is taken to relieve or avoid withdrawal symptoms.

Patients with a high utilization of inpatient detoxification having two or more inpatient detoxifications within a 6 month period immediately preceding treatment are a subset of patients with severe alcohol use disorder. An inpatient detoxification is defined as detoxification in a clinical setting wherein the individual lives in the facility for a period of at least 3 days. In general, inpatient detoxification includes daily monitoring by a physician, 24-hour nursing care, administration of medications to treatment withdrawal symptoms, and careful monitoring of with withdrawal symptoms and vital signs. Patients with severe alcohol use disorder frequently have history of severe withdrawal including delirium tremens (D.T.'s) and/or withdrawal seizures which may require admission to an Intensive Care Unit and other interventions such as anesthesia and mechanical ventilation. In an embodiment, the patient has had two to seven or more inpatient detoxifications in a 6 month period.

As used herein, daily administering includes both self-administration of the medications, as observed dosing of the medications, or administration within a clinical setting. For example, a patient may have a one-week supply of medication that is self-administered on a daily basis. In order to improve patient compliance, administration may be observed by a third party one, twice or more per week. For example, the patient may bring the medications to a clinic for observed dosing. Alternatively, observed dosing and/or daily dispensing of medications may occur on a daily basis to ensure patient compliance.

As used herein an amount sufficient to reduce or eliminate cravings for alcohol and maintain sobriety is an amount of disulfiram and an amount of benzodiazepine that, when combined, reduce or eliminate cravings for alcohol based on the Penn Alcohol Cravings Scale and maintain patient sobriety allowing the patient to function on an outpatient basis. Of note, the American Psychiatric Association recent added cravings as part of the diagnostic criteria for alcohol use disorder. This is based on a large body of evidence which shows that cravings for alcohol are an important indicator of disease severity, causing impairment in patients' overall functioning, and increases risk for relapse. Therefore, medications which reduce cravings have clinical significance. The amount of disulfiram is 250-500 mg mg daily and the amount of benzodiazepine is the equivalent of 1 to 3 mg of clonazepam daily. Equivalents to clonazepam are given in the table:

| | | |
|---|---|---|
| Clonazepam | 1 | mg |
| Alprazolam | 0.5 | mg |
| Lorazepam | 2 | mg |
| Diazepam | 5 | mg |
| Flurazepam | 15 | mg |
| Triazolam | 0.5 | mg |
| Chlordiazepoxide | 25 | mg |

For diazepam, the daily dose is 5 to 15 mg, which is significantly lower than the dose taught in U.S. Pat. No. 5,140,032 of 20-40 mg of diazepam per day for the average patient, and the expected daily dose of 20 mg per day of diazepam taught in WO 2004/054570. In an embodiment, the patient is not coadministered an antidepressant or an antipsychotic medication as standard practice, however, patients may have co-occurring mental disorders and thereby are treated with other psychiatric medication however these medications are not considered as directly related to treatment of alcohol use disorder or this treatment protocol.

In an embodiment, a reduction in cravings for alcohol is measured as 0-30 on the Penn Alcohol Cravings Scale (PACS). The PACS is a 5-item self-rated scale designed to assess alcohol craving severity (frequency, intensity, duration, resistance, and overall craving) during the preceding 1 week. Each item has a score range of 0-6 (maximum total craving score=30). The PACS has demonstrated excellent reliability and good construct/discriminant/predictive validity. Individuals suffering from severe alcohol use disorder with high utilization of inpatient detoxification of have PACS value of 20-30/30. Mild cravings are defined as a value of 0-5/30 on the PACS. The PACS is explained in the Table:

1. During the past week, how often have you thought about drinking or about how good
a drink would make you feel?

| | |
|---|---|
| 0 | Never (0 times during the past week) |
| 1 | Rarely (1 to 2 times during the past week) |
| 2 | Occasionally (3 to 4 times during the past week) |
| 3 | Sometimes (5 to 10 times during the past week or 1 to 2 times per day) |
| 4 | Often (11 to 20 times during the past week or 2 to 3 times per day) |
| 5 | Most of the time (20 to 40 times during the past week or 3 to 6 times per day) |
| 6 | Nearly all of the time (more than 40 times during the past week or more than 6 |
| | times per day) |

2. At its most severe point, how strong was your craving during the past week?

| | |
|---|---|
| 0 | None at all |
| 1 | Slight, that is, a very mild urge |
| 2 | Mild urge |
| 3 | Moderate urge |
| 4 | Strong urge, but easily controlled |
| 5 | Strong urge and difficult to control |
| 6 | Strong urge and would have drunk alcohol if it were available |

3. During the past week, how much time have you spent thinking about drinking or
about how good a drink would make you feel?

| | |
|---|---|
| 0 | None at all |
| 1 | Less than 20 minutes |
| 2 | 21 to 45 minutes |
| 3 | 46 to 90 minutes |
| 4 | 90 minutes to 3 hours |
| 5 | Between 3 to 6 hours |
| 6 | More than 6 hours |

4. During the past week, how difficult would it have been to resist taking a drink if you
had known a bottle were in your house?

| | |
|---|---|
| 0 | Not difficult at all |
| 1 | Very mildly difficult |
| 2 | Mildly difficult |
| 3 | Moderately difficult |
| 4 | Very difficult |
| 5 | Extremely difficult |
| 6 | Would not be able to resist |

5. Keeping in mind your responses to the previous questions, please rate your overall
average alcohol craving for the past week.

| | |
|---|---|
| 0 | Never thought about drinking and never had the urge to drink |
| 1 | Rarely thought about drinking and rarely had the urge to drink |
| 2 | Occasionally thought about drinking and occasionally had the urge to drink |
| 3 | Sometimes thought about drinking and sometimes had the urge to drink |
| 4 | Often thought about drinking and often had the urge to drink |
| 5 | Thought about drinking most of the time and had the urge to drink |
| | most of the time |
| 6 | Thought about drinking nearly all of the time and had the urge to drink |
| | nearly all of the time |

In an embodiment, daily administration of the combination of disulfiram and a benzodiazepine increases the ability of the individual to engage in outpatient substance abuse psychotherapy. Participating in substance abuse psychotherapy provides patients the opportunities to learn about psychological or social factors which may affect likelihood of relapse such as stress tolerance, environment triggers (such as seeing other people consume alcohol), and behavioral warning signs for relapse (such as social isolation or purposefully driving by a liquor store without entering).

In an embodiment, abstinence from alcohol is maintained for at least 3 months, 6 months, one year, five years, and longer.

Disulfiram inhibits alcohol dehydrogenase and thereby causes build-up of acetaldehyde when combined with alcohol. The risk of the negative effects of excessive acetaldehyde promotes sobriety through negative reinforcement. Benzodiazepines act similar to alcohol in that they stimulate GABA receptors. However, there is significant risk when benzodiazepines are combined with alcohol. Without being held to theory, it is believed that the combination of disulfiram and a benzodiazepine acts a safe agonist maintenance therapy for alcohol use disorder because benzodiazepine stimulates similar neuroreceptors as alcohol and disulfiram further reinforces sobriety through risk of reaction with alcohol. Agonist maintenance treatment has been used successfully in the treatment of dependence of various substances including opioids, nicotine, and recently cocaine. It is considered the standard of care for patients with severe substance use disorders who are unable to sustain sobriety through other means such as participation in 12-step programs or substance abuse psychotherapy.

The disulfiram and the benzodiazepine are administered in the form of a pharmaceutical composition containing a pharmaceutically acceptable excipient such as a composition for oral administration. Oral pharmaceutical compositions may be may be enclosed in hard or soft shell gelatin capsules or may be compressed into tablets. For oral therapeutic administration, disulfiram and the benzodiazepine may be combined with one or more excipients and used in the form of ingestible tablets, buccal tablets, troches, capsules, elixirs, suspensions, syrups, wafers, and the like. The disulfiram and the benzodiazepine may be administered as separate compositions, or as a combination dosage form containing both active agents.

The tablets, troches, pills, capsules, and the like may also contain binders, disintegrating agents, lubricants, and sweetening agents. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain N-acetylcysteine, sucrose or fructose as a sweetening agent, methyl and propylparabens as preservatives, a dye and flavoring such as cherry or orange flavor. In addition, disulfiram and the benzodiazepine may be incorporated into extended or sustained-release preparations and devices.

The invention is further illustrated by the following non-limiting examples.

### EXAMPLES

### Methods

10 patients with history of severe alcohol use disorder with an average of 4 inpatient detoxifications within the past six months were administered 250 mg of disulfiram as well as a benzodiazepine (clonazepam 1 mg TID).

### Results

The results of the study are given in the Table 1. 7 out of 10 patients maintained sobriety throughout the subsequent 3 months after starting protocol. Of the 3 patients who relapsed, length of time until relapse ranged from 5 to 51 days with mean average being 29.3 days. Only one of the participants required inpatient detoxification during study period.

| | Number of | | Number of |
|---|---|---|---|
| | inpatient | | inpatient |
| | detoxifications | | detoxifications |
| | within last | Length of Time | within 3 months after |
| | 6 month | before relapse | starting treatment |
| Patient 1 | 3 | 5 days | none |
| Patient 2 | 5 | None | none |
| Patient 3 | 5 | None | none |
| Patient 4 | 7 | 32 days | none |
| Patient 5 | 3 | None | none |
| Patient 6 | 2 | none | none |
| Patient 7 | 4 | none | none |
| Patient 8 | 5 | none | none |
| Patient 9 | 2 | none | none |
| Patient 10 | 4 | 51 days | 1 |
| Average: | mean average 4 | mean average | Range 0-1 |
| | (standard | 29.3 days | |
| | deviation 5.65) | (for patients | |
| | | who did relapse) | |
| | | (most patients | |
| | | did not) | |

The study also showed decrease in cravings for alcohol with patients reporting between 0-5/30 on PACS one week after initiating treatment.

The use of the terms “a” and “an” and “the” and similar referents (especially in the context of the following claims) are to be construed to cover both the singular and the plural, unless otherwise indicated herein or clearly contradicted by context. The terms first, second etc. as used herein are not meant to denote any particular ordering, but simply for convenience to denote a plurality of, for example, layers. The terms “comprising”, “having”, “including”, and “containing” are to be construed as open-ended terms (i.e., meaning “including, but not limited to”) unless otherwise noted. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof Therefore, it is intended that the invention not be limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context.

1. A method of decreasing a recurrence of hospitalization of an individual suffering from severe alcohol use disorder with high utilization of inpatient detoxification, comprising
administering daily to the individual a combination of disulfiram and a benzodiazepine in amounts sufficient to reduce or eliminate cravings for alcohol and maintain sobriety,
wherein severe alcohol use disorder with high utilization of inpatient detoxification is defined as two or more inpatient detoxifications within a 6 month period immediately preceding treatment, and
wherein a decrease in the recurrence of hospitalization is one or less hospitalizations in the 3 month period following the start of treatment.

2. The method of claim 1, wherein administering is performed on an outpatient basis.

3. The method of claim 1, wherein the daily dose of disulfiram is 200-500 mg daily and the dose of benzodiazepine is equivalent to clonazepam 1 to 3 mg daily.

4. The method of claim 1, wherein the diazepam and the benzodiazepine are not coadministered with an antidepressant or an antipsychotic medication.

5. The method of claim 1, wherein the benzodiazepine is clonazepam, lorezapam, diazepam, alprazolam, chlordiazepoxide, fluorezepam, or triazolam.

6. The method of claim 1, further comprising measuring the recovery of the individual on a Penn Alcohol Cravings Scale (PACS) of 0-30.

7. The method of claim 6, wherein prior to treatment, the individual has a Penn Alcohol Cravings Scale value of 20-30/30.

8. The method of claim 7, wherein within one week of daily administration of the combination of disulfiram and benzodiazepine the individual has Penn Alcohol Cravings Scale value of 0-5/30.

9. The method of claim 1, wherein daily administration of the combination of disulfiram and benzodiazepine increases the ability of the individual to engage in outpatient substance abuse psychotherapy.

10. The method of claim 1, wherein abstinence from alcohol is maintained for at least three months.

11. A method of decreasing cravings for alcohol in an individual suffering from severe alcohol use disorder with high utilization of inpatient detoxification, comprising
administering daily to the individual a combination of disulfiram and a benzodiazepine in amounts sufficient to reduce or eliminate cravings for alcohol and to maintain sobriety,
wherein severe alcohol use disorder with high utilization of inpatient detoxification is defined as two or more inpatient detoxifications within a 6 month period immediately preceding treatment, and
wherein cravings for alcohol are measured on a Penn Alcohol Cravings Scale of 0 to 30.

12. The method of claim 11, wherein administering is performed on an outpatient basis.

13. The method of claim 11, wherein the daily dose of disulfiram is 200-500 mg daily and the dose of benzodiazepine is equivalent to clonazepam 1 to 3 mg daily.

14. The method of claim 11, wherein the diazepam and the benzodiazepine are not coadministered with an antidepressant or an antipsychotic medication.

15. The method of claim 10, wherein the benzodiazepine is clonazepam, lorezapam, diazepam, alprazolam, chlordiazepoxide, fluorezepam, or triazolam.

16. The method of claim 11, wherein prior to treatment, the individual has a Penn Alcohol Cravings Scale value of 20-30/30.

17. The method of claim 17, wherein within one week of daily administration of the combination of disulfiram and benzodiazepine the individual has Penn Alcohol Cravings Scale value of 0-5/30.

18. The method of claim 1, wherein daily administration of the combination of disulfiram and benzodiazepine increases the ability of the individual to engage in outpatient substance abuse psychotherapy.

19. The method of claim 1, wherein abstinence from alcohol is maintained for at least three months.
